(19) United States
(12) Patent Application Publication
(10) Pub. No.: US 2007/0016087 A1
(43) Pub. Date: Jan. 18, 2007

(54) Method for acquisition and display of non-invasive blood pressure

(76) Inventor: Paul Richard Voith, Cedarburg, WI (US)

Correspondence Address: PAUL VOITH, 10387 HIGNLAWN CT., CEDARBURG, WI, 53012, US

(21) Appl. No.: 11/427,905

(22) Filed: Jun. 30, 2006

Related U.S. Application Data
(60) Provisional application No. 60/695,812, filed on Jul. 1, 2005.

(51) Int. Cl. A61B 5/02

(52) U.S. Cl. 600/493; 600/485

(57) ABSTRACT

A motion tolerant blood pressure measuring system including means to record cuff pressure, Korotkoff sounds and heart beat and to display them in relation to each other. A processor digitizes and correlates the three signals to display the relationship between the occurrence of a Korotkoff sound, the pressure at which it occurred, and the time delay from the start of a cardiac cycle. Positions representing valid Korotkoff sounds form a continuous curve which is substantially representative of the rising edge of the subject's pulse pressure waveform. Indications not collocated with such a curve may be identified as artifact both visually and algorithmically. The operator, using the natural human ability to quickly visualize points forming a continuous line, may compare such observations with the results determined by the algorithm to instantly assess algorithmic validity.

### BACKGROUND OF THE INVENTION

Automated means exist for determining human blood pressure. They can be divided broadly into two categories, invasive and non-invasive. Invasive measurements require some portion of the instrument to reside within an artery. Invasive measurements are almost always continuous in nature; displaying a waveform which shows the pressure rise and fall within and during each heart beat. Non-invasive measurements may be either continuous (tonography for example) providing waveform data similar to invasive means, semi-continuous (pulse wave velocity for example) providing discrete values for every heart beat which may be trended into a waveform, or non-continuous (oscillometry for example) providing systolic, diastolic and mean arterial pressure values as discrete numbers representing the maxima, minima and mean of the pressure waveform captured over a series of heart beats displayed at a rate of perhaps once in several minutes.

Most automated non-continuous devices are based primarily on one of two methods; oscillometry or auscultation. Both methods involve the use of an occluding cuff which encircles the subject's limb (usually the upper arm) and is inflated to a pressure sufficient to occlude arterial blood flow in the limb. The pressure in the cuff is then reduced slowly so as to observe certain parameters as arterial blood flow resumes. In the case of oscillometry the observed parameter is the magnitude of oscillations in the cuff pressure due to small volume changes which result from pulsatile blood flow within the encircled limb. It has been found that such oscillations are maximal when the pressure in the cuff matches the average arterial pressure of the encircled limb (called Mean Arterial Pressure or MAP). Systolic and diastolic pressure values are determined from an analysis of the oscillations recorded at pressures higher and lower than the mean. The heart rate may be provided by analyzing the repetition rate of the oscillations which occur once per heart beat. In the case of auscultation the observed parameter is the acoustic (and sub acoustic) momentary vibrations which occur distal to the center of the occluding cuff measured at the skin over the primary artery in the encircled limb each time the heart beats while the cuff is between the systolic and diastolic arterial blood pressure. When the cuff pressure is above the highest arterial pressure the artery remains closed and no sound is generated. When the cuff pressure is below the lowest arterial pressure the artery remains open throughout the entire cardiac cycle and no sound is generated. When performed manually by a human observer a stethoscope is used to detect the vibrations and they are referred to as Korotkoff sounds (named for their discoverer). When performed by an automated device a contact microphone or other transduction device may be employed to detect the vibrations. In any case we shall refer to such vibrations as Korotkoff sounds even if the method of transduction or the frequency content would place it outside the range normally considered acoustic. We shall use the term Korotkoff indication to refer to a Korotkoff sound detection as reported by automated equipment; this may include some detections which are artifactual in nature and thus do not represent Korotkoff sounds.

Also in auscultation type systems it is known that the Korotkoff sound is generated in each cardiac cycle at the instant when the pressure in the compressed artery rises above the pressure of the occluding cuff allowing blood to flow. Sounds are generated only when the cuff pressure is between the highest (systolic) and lowest (diastolic) pressure as the rising arterial pressure forces the artery open. If a means for determining the subject's heart beat is available (using the electrocardiogram for example) then an algorithm may be improved by using a method which rejects, as artifact, those Korotkoff indications which are either to early or too late to have been generated by the arterial pressure rise. This method is sometimes referred to as R-wave gating since the fiducial point chosen in the electrocardiogram as the reference for the start of the cardiac cycle is often the so called “R wave” generated when the heart ventricles contract.

Under certain circumstances it is desirable to obtain blood pressure readings while the subject is undergoing significant physical activity, as occurs, for example, during a medical stress test. During such tests the subject's cardiovascular system is intentionally challenged by, for example, running on a treadmill. For safety reasons it is necessary for medical personnel to continually monitor the subject's blood pressure since a sudden rise or fall in blood pressure may warrant the early termination of the stress test and may indicate the need for immediate medical intervention.

Unfortunately the physical motion which accompanies the stress test tends to render current automated blood pressure recording devices unreliable. As a result the most widely used blood pressure recording means during treadmill stress testing is manual auscultation using an inflatable cuff and stethoscope. This places a burden on the medical personnel who must manage to perform this task while the subject is in motion or else momentarily stop the subject to obtain a still reading. In the former case the moving cuff and stethoscope inject considerable acoustic noise which makes manual auscultation problematic. The subject must run in an awkward manner with arm raised and with range of motion limited. Medical personnel must divert attention from other critical monitoring to perform this labor intensive task. In the later case the subject must step off the moving portion of the treadmill used in the test or the treadmill must be stopped and restarted. This can result in falls and injuries which can be especially severe in elderly subjects who make up a large population of those undergoing such testing. As a minimum the continuity of the test, which is intended to create a continuous progression of stress, is disrupted for minutes at a time.

In response to this need some automated devices have been developed which are intended to be more motion tolerant than devices designed for use on still subjects. These are almost exclusively of the auscultation type described above perhaps including R-wave gating to mitigate the negative effect of motion artifact. Other methods including invasive, non-invasive continuous and oscillometry based methods have been found to be too sensitive to motion artifact to be useful in this application. The motion tolerant automated auscultation type devices typically display a simple numeric result consisting of a pair of values representing the systolic and diastolic pressure values and additionally may include heart rate and perhaps other discrete values.

Even though these devices are considered motion tolerant in comparison to other technologies they suffer from a number of deficiencies which it is the object of the present invention to overcome. Stress tests in particular tend to be progressive in nature so that a device which provides reasonable accuracy at the start of a test may begin to become increasingly compromised as the test progresses. Factors which affect reliability of the result include motion artifact appearing as possible Korotkoff sound detections, rising and falling cuff pressures affecting the distribution of Korotkoff detections, periodic foot fall artifact matching the period of the heart beat, a change in blood pressure during a reading, and others. Automated auscultatory algorithms have particular difficulty with artifactual Korotkoff indications just above the actual systolic blood pressure or just below the actual diastolic pressure.

On existing devices there is no mechanism for the operator to objectively assess the factors which compromise the validity of a result other than to have the device simply report that the result as valid or invalid. A typical reporting mechanism is to inhibit the display of results which are determined to be invalid. A device may provide a scoring value representing a continuum of quality level (a percentage value indicating the relative reliability of the result for example) but since the operator has no means to know upon what criteria the score is based in a particular instance it is of limited value. Moreover, if the device were to indicate, for example, 75% reliable result, it is not obvious if the operator should accept the result or not. If a criterion is established at what level to accept the result then the presentation of the level is inconsequential and the device may as well report simply valid or invalid. If the device displays a result as valid the clinician has no method to secondarily determine if the result is plausible other than performing a manual auscultation. Likewise if the device fails to display a result the clinician must begin manual auscultation with no easy method for determining if the quality would have been sufficient.

### SUMMARY OF THE INVENTION

It is a the primary object of the invention to provide a new and improved method for processing and display of blood pressure data suitable for use on subjects in motion.

It is further an object of the invention to provide the clinician an objective indication of the quality of the data used to generate the result.

It is a further object of the invention to provide the clinician a means to independently estimate the blood pressure values from partially processed data.

It is further an object of the invention to provide display of the data which allows signals representing Korotkoff sounds to be easily discriminated from spurious noise pulses.

It is further an object of the invention to provide a means to determine if the blood pressure is undergoing change.

It is further an object of the invention to provide a means to reduce the negative affect of motion induced changes in the occluding cuff pressure.

These and other objects and advantages of the present invention will become more apparent from the detailed description thereof taken with the accompanying drawings.

In general terms, the invention comprises a transducer for use in detecting Korotkoff sounds, a means for applying pressure to the artery for preventing blood flow including means for reducing the pressure on the artery whereby blood flow commences when the pressure applied to the artery falls below the subject's systolic pressure. The invention also includes a means to determine the time delay between a fixed point in the cardiac cycle and the occurrence of the Korotkoff sound. The invention includes a signal processor to determine the validity of the Korotkoff detections, their relative position in the cardiac cycle, and the cuff pressure at which the detections occur. The invention also includes a device suitable for display of a plurality of Korotkoff indications in such a manner as allows the time of occurrence within the cardiac cycle and coincident cuff pressure to be intuited. Such a display may, for example, use a particular icon for each of the displayed Korotkoff indications whose horizontal position upon a graph indicates the time of occurrence within the cardiac cycle and whose vertical displacement indicates the coincident cuff pressure.

### BRIEF DESCRIPTION OF THE INVENTION

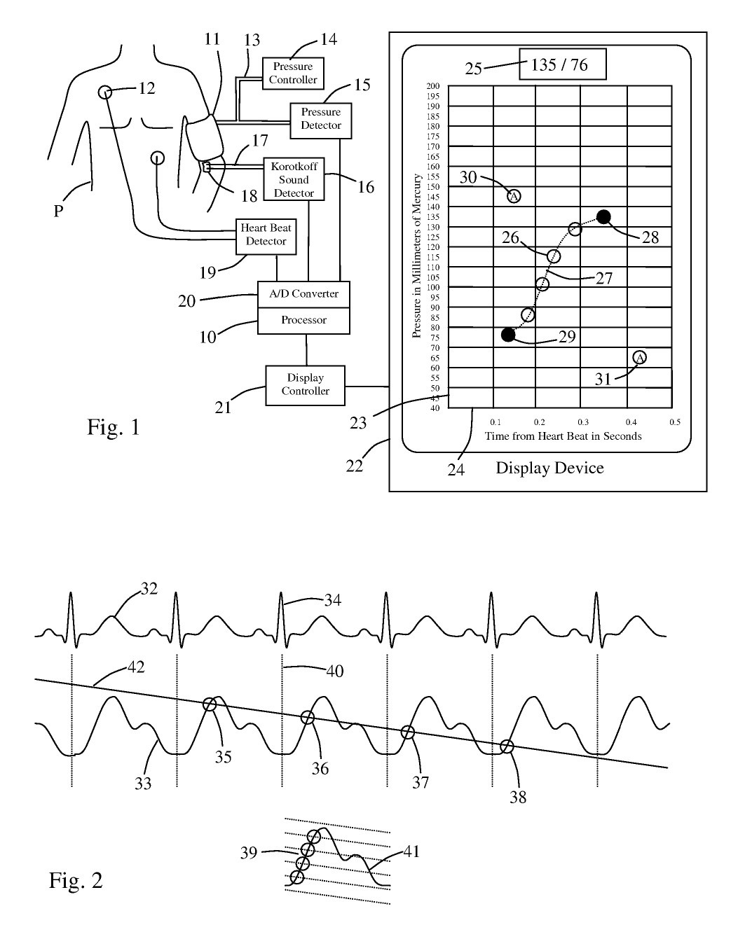

FIG. 1 schematically illustrates a blood pressure recording and display system.

FIG. 2 illustrates the method by which an electrocardiogram is used to partition the arterial blood pressure waveform into distinct beats whose Korotkoff indications may then be superimposed.

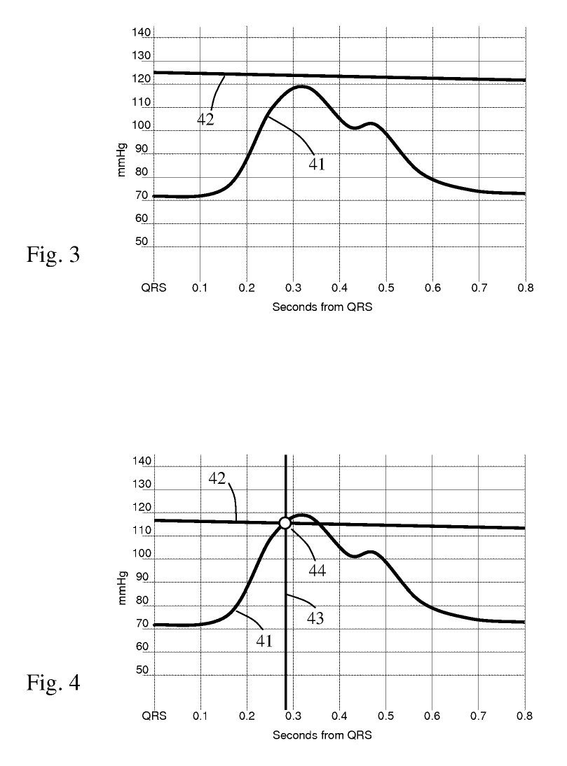

FIG. 3 shows a plot of the arterial pulse pressure and cuff pressure waveforms versus time from start of cardiac cycle and cuff pressure.

FIG. 4 shows a plot of the arterial pulse pressure and its intersection with cuff pressure waveforms versus time from start of cardiac cycle and cuff pressure.

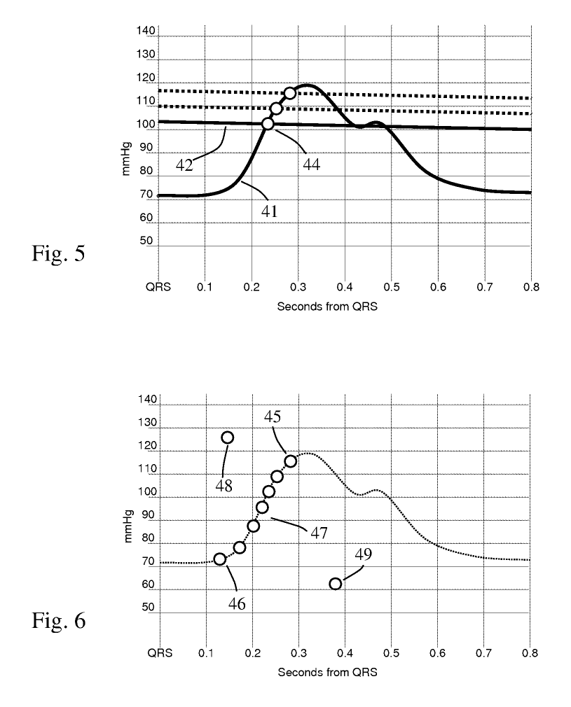

FIG. 5 shows a plot of a sequence of intersections of the type shown in FIG. 3.

FIG. 6 shows a plot of a plurality of intersections, representing Korotkoff sounds as compared to an arterial pulse pressure waveform.

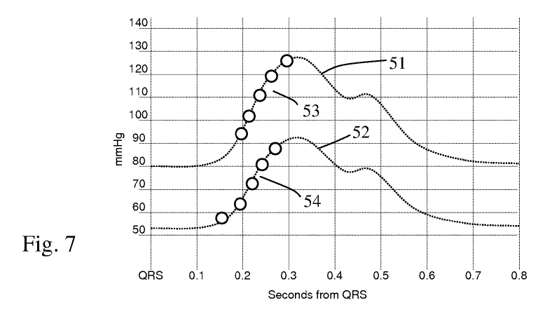

FIG. 7 shows a plot of a plurality Korotkoff sounds as the arterial pulse pressure changes.

### DETAILED DESCRIPTION OF THE PREFERRED EMBODIMENTS

The pressure measuring system according to the invention is shown in FIG. 1 to include a transducer 18 which is mounted towards the distal end of a pressure cuff 11 and coupled to a Korotkoff sound detector 16 using a suitable cable 17. The Korotkoff sound detector is connected to an analog to digital converter 20. The pressure cuff 11 is conventional and will not be described in detail for the sake of brevity. It will be sufficient for the purpose of understanding the invention to state that the cuff 11 comprises a bladder constructed and arranged to be mounted on the arm P of a patient in a conventional manner. A pressure controller 14 is connected to the cuff 11 via manifold 13 for inflating the cuff to a pressure above the patient's systolic pressure and for reducing the pressure in cuff 11 to below the patient's diastolic pressure. The pressure controller 14 may be of conventional design for both sourcing pressure and relieving pressure at a controlled rate and will not be discussed in detail for the sake of brevity. A pressure detector 15 is also attached to the cuff via manifold 13 and converts the pressure signal to an electrical voltage is connected to analog to digital converter 20. A heart beat detector 19 is connected to a plurality of electrodes 12 for obtaining an electro cardiograph signal from which the onset of a heart beat may be determined. This signal is connected to analog to digital converter 20. The analog to digital converter 20 is connected to processor 10 which correlates the Korotkoff sound detections with the cuff pressure and the onset of the heart beat to produce a graph which is output to the display controller 21 which drives the display device 22. The resulting display provides a graph showing the position of the Korotkoff indications 26 plotted against the time from the onset of the prior heartbeat on the horizontal axis 24 and against the cuff pressure at which the sound was detected plotted on the vertical axis 23.

The data representing valid Korotkoff detections will substantially form a line 27 which has a maximum 28 and a minimum 29. The pressure at which the maximum occurs is the systolic blood pressure while the pressure at which the minimum occurs is the diastolic blood pressure. These are interpreted by the processor 10 and displayed as the automated result 25 shown in FIG. 1 by the common method of displaying the systolic value over the diastolic value. Artifactual Korotkoff detections 30, 31 are substantially distant from the line 27 formed by the valid Korotkoff detections. These are visible to the operator at the same time as the result and so the operator is free to determine if the machine result is reasonable given the relative positions of the artifactual detections.

FIG. 2 illustrates the method by which an electrocardiogram (ECG) 32 may be used to partition the arterial blood pressure waveform 33 into distinct beats. An easily identifiable periodic fiducial point is selected on the ECG, such as the largest deflection of the QRS complex 34 of each heart contraction, to become the reference 40 for the start of partition. Because the arterial pressure wave form is generated by the heart its time relationship to the QRS is constant. Successive Korotkoff indications 35, 36, 37, 38 which occur at the intersection of the gradually decreasing cuff pressure 42 and the rising edge of the arterial pressure waveform 33 may be superimposed to form a single graph containing a plurality of Korotkoff indications 39 which substantially conform to the rising edge of a representative arterial waveform 40 spanning a single heartbeat.

The graph in FIG. 3 displays pressure in millimeters of mercury (mmHg) on the vertical axis and seconds from the onset of the heartbeat on the horizontal axis. The pressure versus time within the occluding cuff is plotted as a line 42 above the arterial pressure whose waveform 41.

FIG. 4 shows that the Korotkoff indication 44 occurs at a time, shown by the vertical line 43, at which the cuff pressure 42 intersects the rising edge of the arterial blood pressure waveform 41.

In FIG. 5 we see that a plurality of such intersections can be obtained by recording the location of each Korotkoff indication on the pressure versus time graph for successive heart beats at progressively lower cuff pressures.

As the cuff pressure is reduced a plurality of indications, shown in FIG. 6, trace out a line conforming to the rising edge of the pulse pressure waveform 47. Korotkoff indications 48, 49 which are not substantially on the line are visually identifiable as artifact. The Korotkoff indication 45 highest in pressure but still conforming to such a line becomes an accurate estimate of the peak of the arterial waveform which is equal to the systolic blood pressure. Likewise the Korotkoff indication 46 at the lowest pressure which substantially conforms to the leading edge of the arterial waveform is an accurate estimate of the diastolic blood pressure.

A changing arterial pressure will appear as two distinct lines of Korotkoff sound detections as shown in FIG. 7. The high pressure waveform 51 has a leading edge which causes Korotkoff indications to conform to a line 53 which is in a different location than that of the leading edge 54 of the lower pressure waveform 52. Normally it is not possible to determine the difference between a pressure which is changing and a pulse pressure which is simply large.

Although the examples provided show a cuff pressure which is slowly changing from high pressure to low pressure an advantage of the invention is that the Korotkoff indications conform to the rising edge of the pulse pressure waveform regardless of whether the cuff pressure is rising or falling or varying in an arbitrary pattern. In addition, even though the examples show a condition in which the displayed Korotkoff indications are from successive heartbeats, this is not a requirement. An advantage of the invention is that Korotkoff indications from non-successive heartbeats will accumulate to form a continuous line pattern as long as the blood pressure is not changing substantially in the time over which the indications are recorded. It should also be understood that although present systems typically operate by inflating and then slowly deflating the cuff to obtain a single reading, it is an advantage of the invention that the Korotkoff indications from several such inflation/deflation cycles may be shown on the display at one time with each indication remaining in view for some pre-determined period of time.

It is also an object of the invention that the algorithmically determined systolic and diastolic values may be presented on the display graph 22 by, for example, distinguishing the icons used to represent the maximal and minimal non-artifactual Korotkoff indications 28, 29. In addition it is an object of the invention that any of the icons representing Korotkoff indications may be varied to distinguish more recent from older indications, the magnitude of the Korotkoff sound which provided the indication, an automated assessment of the quality of the source data used to provide the indication, or an automated assessment that the indication is artifactual.

I claim:

1. A blood pressure measuring system including a transducer applied to the subject in an opposed relation to an artery, means for detecting signals from said transducer corresponding to Korotkoff sounds, means for applying pressure to the artery for preventing blood flow, means for raising and lowering the pressure applied to the artery, means for recording the applied pressure, means for detecting a heartbeat, processing means connected to said pressure recoding means, Korotkoff sound detection means, and heartbeat detection means, said processing means being connected to a display means capable of displaying a graph, said graph including icons for Korotkoff indications displayed in relation to pressure and time from heart beat detection.

1. The system set forth in claim 1 wherein the icons indicating Korotkoff indications are varied according to the time since recording.

2. The system set forth in claim 1 wherein the icons indicating Korotkoff indications are varied according to magnitude.

3. The system set forth in claim 1 wherein the icons indicating Korotkoff indications are varied according to data quality or on account of having been determined artifactual.

4. The system set forth in claim 1 wherein the icons indicating Korotkoff indications are varied or otherwise distinguished as being the maximal (systolic) or minimal (diastolic) pressure values.

5. The system set forth in claim 1 wherein an automatically calculated estimate of the systolic and diastolic blood pressure values is displayed.

6. The system set forth in claim 1 wherein the onset of the heartbeat is determined from and electrocardiogram signal.
